(19) United States
(12) Patent Application Publication
(10) Pub. No.: US 2024/0366410 A1
(43) Pub. Date: Nov. 7, 2024

(54) SYSTEMS AND METHODS FOR COLLAPSING AND LOADING A PROSTHETIC DEVICE

(71) Applicant: Corcym S.r.l., Milan (IT)

(72) Inventors: Giovanni GIORDANO, Chivasso (IT); Monica Francesca ACHILUZZI, Chivasso (IT)

(73) Assignee: Corcym S.r.l., Milan (IT)

(21) Appl. No.: 18/292,863

(22) PCT Filed: Jul. 29, 2021
(86) PCT No.: PCT/IB2021/056942

§ 371 Date: Jan. 26, 2024

(51) Int. Cl. A61F 2/95; A61F 2/24

(52) U.S. Cl. CPC A61F 2/9524; A61F 2/2427

(57) ABSTRACT

Systems and methods are provided for collapsing and loading a prosthetic device for delivery to a patient. The collapsing and loading systems described herein may facilitate more efficient and consistent positioning of a prosthetic device with respect to a collapser system and a corresponding delivery system. For example, the system may include a collapser system having a base and a collapser movably coupled to the base. The collapser defines an orifice sized and shaped to receive a prosthetic device (e.g., a prosthetic heart valve) in a first position and may be moved relative to the base to a second position for loading and collapsing the prosthetic device onto a delivery system.

### FIELD OF USE

This disclosure generally relates to systems and methods for manipulating implantable prosthetic devices, such as heart valve prostheses, during preoperative procedures preceding the implantation of such devices.

### BACKGROUND

In the fields of heart valve surgery and interventional cardiology, easy handling of medical devices and reduction of the time required to perform safe and accurate surgical interventions and procedures are topics of central interest for medical and technological research. With reference to the implantation of expandable heart valve prostheses such as sutureless heart valve prostheses, current practice provides that heart valve prostheses may need to be radially collapsed and coupled to a delivery instrument in order to be delivered to an implantation site of a patient, for example, in a minimally invasive or percutaneous procedure. Collapsing an implantable prosthetic device may present a number of important issues. While devices have been devised to facilitate the collapsing operation, the operation may be rather tenuous and complex to perform.

Examples of previously-known systems for collapsing prosthetic devices, such as heart valve prostheses, are described in U.S. Pat. No. 8,006,535 to Righini, U.S. Pat. No. 9,114,010 to Gaschino, U.S. Pat. No. 9,788,931 to Giordano, and U.S. Patent App. Pub. No. 2021/0196442 to Giordano, each assigned to the assignee of the present invention, the entire contents of each of which are incorporated herein by reference.

One challenge for the practitioner when collapsing a prosthetic device onto a delivery system lies in achieving desired positioning, in particular, angular and longitudinal positioning, of the prosthetic device with respect to both the collapsing system and the related delivery system. In varying prior art embodiments, delivery systems may offer indicia (e.g., markers intended to identify the commissures of an aortic or tricuspid valve) to aid the practitioner in correctly positioning the prosthetic device at the implantation site. These embodiments, however, may involve a risk of damaging the prosthetic device as the practitioner adjusts the angular position of the prosthetic device within the orifice of the collapsing system by directly contacting and manipulating the prosthetic device. Further, these embodiments still often require manual and potentially time-consuming orientation of the prosthetic device with respect to other system components during the collapsing operation. Moreover, systems may not simultaneously facilitate axial and longitudinal alignment of the prosthetic device with respect to both the collapsing and delivery systems.

Another challenge lies in maintaining the prosthetic device in a progressively collapsed state as the collapsing operating proceeds from start to end. While current systems include multistep collapsing operations, the systems may not adequately maintain the prosthetic device in a collapsed configuration on the delivery system.

Yet another challenge involves the problematic system configurations often required for initial loading of a prosthetic device into the collapser system. In varying prior art embodiments, the prosthetic device may only be loaded from a side of the collapser system, which may further require use of an additional holder device.

In view of the foregoing, it would be desirable to provide systems and methods for collapsing and loading a prosthetic device for delivery to a patient, while facilitating more efficient and consistent positioning of the prosthetic device with respect to a collapser system and a related delivery system.

It would further be desirable to provide systems and methods for ensuring the prosthetic device remains collapsed onto the delivery system following use of the collapser system.

It would further still be desirable to provide systems and methods for simplified, user-friendly loading of the prosthetic device into the collapser system.

### SUMMARY

Systems and methods are provided herein for collapsing and loading a prosthetic device in preparation for delivery to a patient. For example, the system may be a kit/pack of accessories provided with the prosthetic device to permit the clinician to implant the prosthetic device. In various embodiments, the present disclosure provides means that render manipulation of implantable prosthetic devices easier, safer, faster, and more accurate in comparison to previously-known systems and methods. For example, systems and methods are provided herein for collapsing and loading a prosthetic device (e.g., a prosthetic heart valve) onto a delivery system in an efficient and consistent manner. Preferably, an exemplary collapser system allows the prosthetic device to remain collapsed onto the delivery system following use of the collapser system to facilitate delivery of the collapsed prosthetic device. The collapser system also permits simplified, user-friendly loading (e.g., top loading) of the prosthetic device into the collapser system and further simplified, user-friendly loading (e.g., side loading) of the delivery system to allow the prosthetic device to be collapsed in preparation for delivery to a target implantation site, such as a defective native heart valve.

In accordance with some aspects, the system may include a collapser system having a base and a collapser movably coupled to the base. The collapser preferably defines an orifice sized and shaped to receive a prosthetic device in an expanded state while the collapser is in a first position (e.g., prosthetic device loading position) relative to the base. The collapser may be configured to move from the first position to a second position (e.g., delivery device loading position) such that the orifice is oriented in a different manner relative to the base than the first position. For example, the collapser system may be designed to permit top loading of the prosthetic device in the first position into the orifice of the collapser, permit movement of the collapser relative to the base from the first position to a second position (e.g., from a horizontal position to a vertical position), and permit side loading of the delivery system into the orifice of the collapser in the second position. The collapser further may be configured to reduce the size of the orifice from a first size to a second, smaller size to collapse the prosthetic device onto a delivery system while the collapser is in the second position to prepare the prosthetic device for delivery to a patient in a collapsed state via the delivery system.

The collapser further may include an actuator configured to, upon actuation, cause the collapser to reduce the size of the orifice from the first size to the second, smaller size such that the prosthetic device is loaded in the collapsed state onto the delivery system. The actuator may be configured to, upon further actuation, cause the collapser to increase the size of the orifice from the second, smaller size to the first size such that the prosthetic device remains collapsed onto the delivery system. In this manner, the delivery system having the collapsed prosthetic device coupled thereto may be withdrawn from the orifice of the collapser to permit implantation of the prosthetic device using the delivery system. The actuator may include a handle to be gripped and moved by a user for actuation. In addition, the collapser system further may include a movable base coupled to the collapser and movably coupled to the base. A portion of the base may extend under the movable base. The collapser further may, in the first position, permit top loading of the prosthetic device in the expanded state into the orifice and, in the second position, permit side loading of the delivery system through the orifice. The first position of the collapser further may be substantially orthogonal to the second position (e.g., horizontal to vertical relative to the base).

The collapser system further may include a lock configured to maintain the collapser in the first position via coupling with the base. The lock may be releasable upon application of a predetermined force towards the second position. The lock further may include a flexible protrusion extending from the base to engage a recess at a top portion of a housing of the collapser. The flexible protrusion may be configured to deflect upon application of the predetermined force to release the recess. The collapser system further may include a ledge extending upwardly from the base. The ledge may be configured to contact and securely maintain the collapser in the first position. The collapser system further may include a base lock configured to maintain the collapser in the second position via coupling with the base.

The collapser system further may include a handle support configured to support a handle of the delivery system while a distal portion of the delivery system is inserted into the orifice for collapsing of the prosthetic device. The handle support further may include alignment features configured to engage with corresponding features of the handle of the delivery system to longitudinally and axially align the delivery system during collapsing of the prosthetic device.

The collapser further may include a housing configured to house first and second collapser sectors, each of which may have a plurality of collapser sector segments. The collapser sector segments may be configured to move, upon actuation, to reduce the size of the orifice from the first size to the second, smaller size. The collapser further may include first and second annular bodies configured for relative rotation about an axis. The first and second annular bodies may each have a plurality of radially spaced curved apertures to receive components of the first and second collapser sectors, respectively. In some embodiments, the plurality of radially spaced curved apertures of the first and second annular bodies are configured to self-lock when the first and second collapser sectors are in a position corresponding to the second, smaller size. A handle further may be coupled to the first and second annular bodies. For example, the handle halves may be integrally formed with the first and second annular bodies, respectively. The collapser further may include a collapser central frame configured to be positioned between the first and second angular bodies in the housing. The collapser further may include a plurality of protrusions that extend into the orifice and define voids therebetween to facilitate to alignment of the prosthetic device into the orifice. The collapser further may prevent components at a distal portion of the delivery system (e.g., sheath and/or distal tip) from fully transitioning to the delivery state when the orifice is reduced to the second, smaller size.

Optionally, the system may include further components beyond the collapser system. For example, the system further may include the delivery system. The system further may include a balloon catheter configured to expand the prosthetic device after deployment from the delivery system. The system further may include the prosthetic device. In a preferred embodiment, the prosthetic device is a prosthetic heart valve.

In accordance with another aspect of the present disclosure, a method is provided for collapsing a prosthetic device for delivery to a patient. The method may include loading a prosthetic device into an orifice of a collapser while the collapser is in a first position relative to a base; moving the collapser from the first position to a second position while the base remains in place; inserting a distal portion of a delivery system through the prosthetic device loaded in the orifice; and collapsing the prosthetic device onto the distal portion of the delivery system via the collapser to prepare the prosthetic device for delivery to a patient in a collapsed state via the delivery system. The method further may include, after collapsing the prosthetic device, actuating the delivery system to move proximal and distal components of the delivery system towards one another to hold proximal and distal ends of the prosthetic device in the collapsed state, and transitioning the collapser from the smaller sized orifice to a larger sized orifice such that the prosthetic device remains coupled to the distal portion of the delivery system in the collapsed state. Transitioning the collapser from the smaller sized orifice to the larger sized orifice further may cause the proximal and distal components of the delivery system to move closer towards one another to securely hold the prosthetic device in the collapsed state for delivery to the patient. The method further may include delivering the collapsed prosthetic device using the delivery system to a target implantation site (e.g., the location of a defective heart valve, which may have been surgically removed) and transitioning the collapsed prosthetic device from the collapsed state to an expanded state to implant the prosthetic device at the target implantation site.

### BRIEF DESCRIPTION OF THE DRAWINGS

FIG. 1 illustrates an exemplary system for collapsing and loading a prosthetic device for delivery to a patient in accordance with some aspects of the present disclosure.

FIGS. 2A to 2F illustrate an exemplary collapser system related to the system for collapsing and loading a prosthetic device for delivery to a patient in accordance with some aspects of the present disclosure.

FIG. 2G illustrates an exemplary annular body of a collapser system that includes structure for self-locking the collapser when the orifice is in the collapsed position.

FIGS. 2H to 2J illustrate various self-locking structures that may be incorporated into the collapser system.

FIGS. 3A to 3F illustrate an exemplary delivery system related to the system for collapsing and loading a prosthetic device for delivery to a patient in accordance with some aspects of the present disclosure.

FIGS. 4A to 4J illustrate an exemplary system and method for collapsing and loading a prosthetic device for delivery to a patient in accordance with some aspects of the present disclosure.

### DETAILED DESCRIPTION

Systems and methods are provided herein for collapsing and loading a prosthetic device for delivery to a patient, and thus may be useful in safely and effectively manipulating implantable prosthetic devices such as heart valve prostheses during preoperative procedures preceding the implantation of such devices. For example, the inventive systems may be designed to collapse a sutureless aortic heart valve prosthesis such as the PERCEVAL™ and PERCEVAL™ PLUS devices commercially available from Corcym S.r.l. (Milan, Italy). Examples of suitable prosthetic devices to be collapsed and delivered using the systems described herein include U.S. Pat. No. 7,857,845 to Stacchino and U.S. Patent App. Pub. No. 2021/0205078 to Carlino, each assigned to the assignee of the present invention, the entire contents of each of which are incorporated herein by reference. The collapsing and loading systems described herein are intended to facilitate more efficient and consistent positioning of a prosthetic device, preferably a heart valve prosthesis, with respect to a collapser system and a related delivery system, ultimately for delivery to a patient. Certain embodiments may further be designed, for example, to ensure the prosthetic device remains collapsed onto the delivery system to a relative degree with respect to the current positioning of the collapser system, and/or to provide for top loading of the prosthetic device into the collapser system while the collapser is in an initial, pre-collapsing orientation, both of which features may facilitate more desirable functionality for the practitioner.

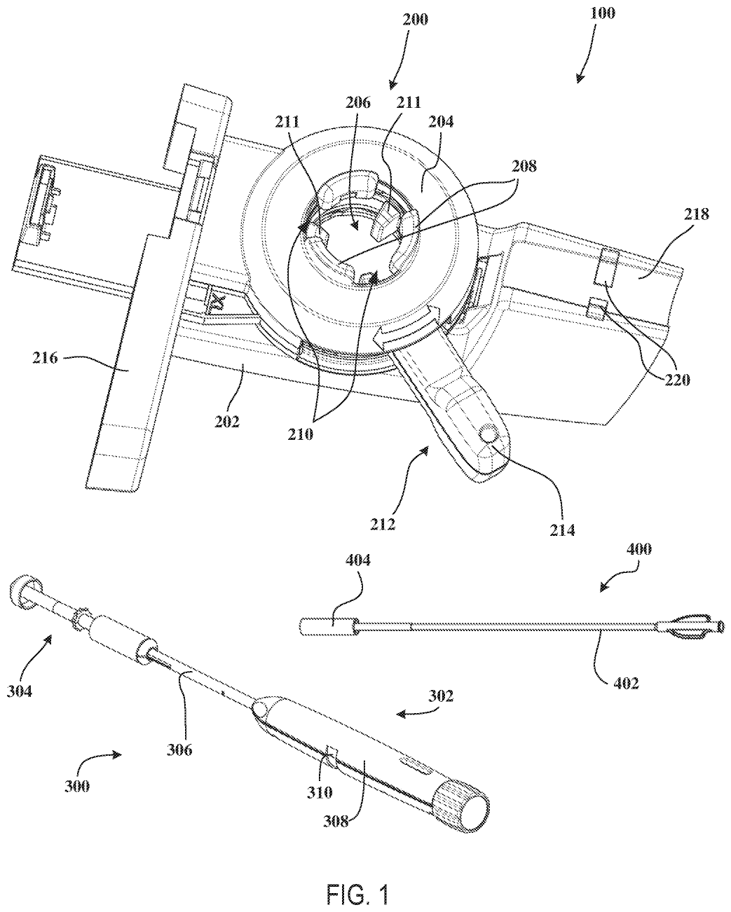

Referring now to FIG. 1, exemplary system 100 for collapsing and loading a prosthetic device for delivery to a patient is provided. System 100 preferably includes collapser system 200 and optionally may further include components for delivering the prosthetic device to a patient such as delivery system 300 and/or balloon catheter 400. In some embodiments, system 100 is a kit/pack of accessories provided with the prosthetic device to permit the clinician to implant the prosthetic device.

As illustrated, collapser system 200 may include base 202 and collapser 204 coupled to base 202. Collapser system 200 generally facilitates collapsing and loading of the prosthetic device onto a delivery system, while base 202 serves as a mounting fixture for collapser 204, the functional center of the collapsing and loading operations. Base 202 permits collapser system 200 to be stably positioned on a surface, such as a table in the operating room, and maintains collapser system 200 in position, even while collapser 204 is moved between positions throughout the loading/collapsing process. Collapser 204 defines orifice 206, which is sized and shaped to receive a prosthetic device in an expanded state. For example, orifice may receive the prosthetic device in an initial, prosthetic loading position as shown in FIG. 1. Collapser 204 is designed to move relative to base 202 to facilitate user friendly, efficient loading of the prosthetic device into orifice 206 in a position and then insertion of the delivery device and collapse of the prosthetic device in a different position(s) relative to base 202. For example, in a second position, orifice 206 is oriented in a different manner relative to base 202 than a first position. Collapser 204 further may reduce the size of orifice 206 from a first size to a second, smaller size to collapse the prosthetic device onto a delivery system, such as exemplary delivery system 300, while collapser 204 is in the second position, thereby preparing the prosthetic device for delivery to a patient in a collapsed state.

Collapser 204 further may include a plurality of protrusions 208 that extend radially inward into orifice 206 and define a plurality of alignment voids 210 therebetween to facilitate alignment of the prosthetic device into orifice 206. Protrusions 208 may also extend longitudinally outward away from orifice 206. In this manner, the prosthetic device may only be loaded into orifice 206 when radially extending portions of the prosthetic device are aligned with voids 210. Protrusions 208 may be radially spaced around orifice 206 in a predetermined manner. For example, there may be three protrusions 208 that are equally spaced (e.g., centers 120° apart) around orifice 206. Collapser 204 further may include a plurality of arm supports 211 that extend radially inward into orifice 206. As illustrated, arm supports 211 may each be positioned at a respective void 210 and are longitudinally offset from the respective void 210. For example, arm supports 211 may be in the central portion of orifice 206, longitudinally-wise whereas protrusions 208 and voids 210 may be at an end of orifice 206, such as the top-loading end in the first position. Like protrusions 208, arm supports 211 may be radially spaced around orifice in a predetermined manner, for example, there may be three arm supports 211 that are equally spaced (e.g., centers 120° apart) around orifice 206. Arm supports 211 are designed to contact radially extending portions of the prosthetic device (e.g., anchoring arms) to facilitate loading of the prosthetic device and collapsing.

Collapser 204 may include actuator 212 configured to, upon actuation, cause collapser 204 to reduce the size of orifice 206 from the first size to the second, smaller size. Actuator 212 may, upon further actuation, cause collapser 204 to increase the size of orifice 206 from the second, smaller size to a larger size, which may be the same size as the first size, such that the prosthetic device remains collapsed onto delivery system 300 and delivery system 300 holding the collapsed prosthetic device may be withdrawn from the collapser. Actuator 212 may take the form of collapser handle 214, which may be held and moved by a user during operation. Alternatively, actuator 212 could be formed from other mechanisms such as a button(s) or the like.

In addition, collapser system 200 further may include moveable base 216 coupled to collapser 204 and moveably coupled to base 202, which may facilitate repositioning of collapser 204 with respect to base 202 as part of the collapsing and loading operations. For example, movable base 216 may be fixed to collapser 204 (e.g., via a central frame) while movable relative to base 202 (e.g., via a hinge). Collapser 204 further may, in the first position, permit top loading of the prosthetic device in the expanded state into orifice 206 and, in the second position, permit side loading of delivery system 300 through orifice 206, as described in further detail below.

System 100 further may include delivery system 300, onto which a prosthetic device may be collapsed and loaded for delivery to an implantation site of a patient by a user. Delivery system 300 may be constructed in a similar manner to the system described in WO2019/224581 (corresponding to U.S. Ser. No. 17/057,502), assigned to the assignee of the present invention, the entire contents of which are incorporated herein by reference. Delivery system 300 may have proximal portion 302 coupled to distal portion 304 via shaft 306. Proximal portion 302 is designed to be positioned outside of the patient during an implantation procedure and manipulated by the user while distal portion 304 is configured to house the collapsed prosthetic device and to be introduced into the patient for implanting the prosthetic device during the procedure. Distal portion 304 is sized to be inserted through the prosthetic device once loaded into orifice 206 for collapsing the prosthetic device onto distal portion 304. Once collapsed, components at distal portion 304 may be manipulated to releasably hold and maintain the prosthetic device in the collapsed state for delivery to the patient. Proximal portion 302 may include handle 308 and/or handle aligners 310. Handle 308 is sized to be held by a user during operation, while handle aligners 310 may be used as temporary fixture points to facilitate longitudinal and axial alignment of delivery system 300 with respect to collapser system 200, as described in further detail below.

Collapser system 200 further may include handle support 218 to support handle 308 of delivery system 300 while distal portion 304 is inserted in orifice 206 for collapsing of the prosthetic device. Handle support 218 may be formed as part of base 202 as illustrated and preferably extends upward a distance such that delivery device 300 remains parallel to the ground contacting portion of base 202 when handle 308 sits and handle support 218 and distal portion 304 sits within the prosthetic device in orifice 206. Handle support 218 may have a geometry corresponding with the geometry of handle 308, such that handle support 218 conformably receives handle 308 in a seated configuration. For example, handle support 218 may have an elongated trough sized to hold the majority of handle 308. Handle support 218 further may include handle support aligners 220, which engage with corresponding handle aligners 310 of handle 308 of delivery system 300 to longitudinally and axially align delivery system 300 during collapsing of the prosthetic device. Handle support aligners 220 may be protrusions extending from a portion of the trough and handle aligners 310 may be recesses to receive the protrusions to longitudinally and axially align delivery system 300.

System 100 further still may include balloon catheter 400 designed to expand the prosthetic device at the implantation site after deployment from the delivery system. Balloon catheter 400 includes catheter 402 and balloon 404 for expanding the prosthetic device as a further step of implantation.

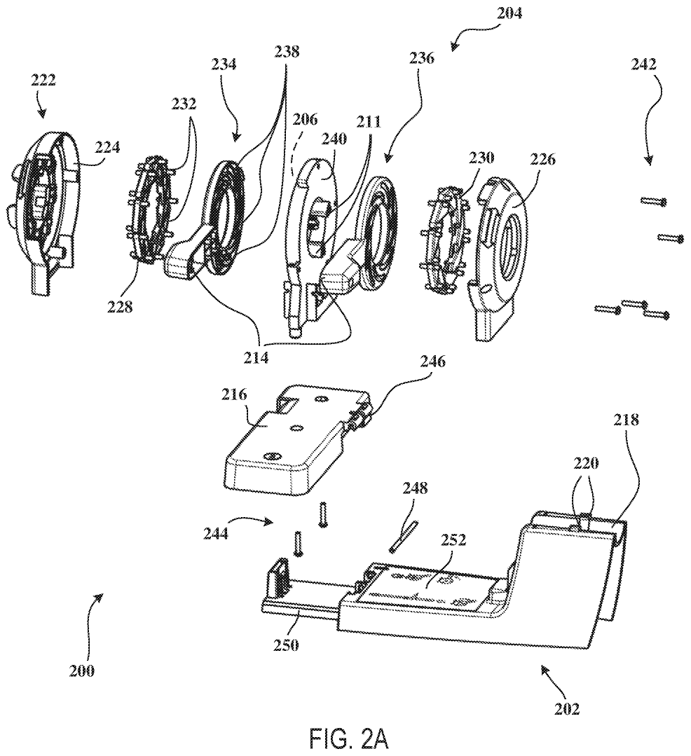

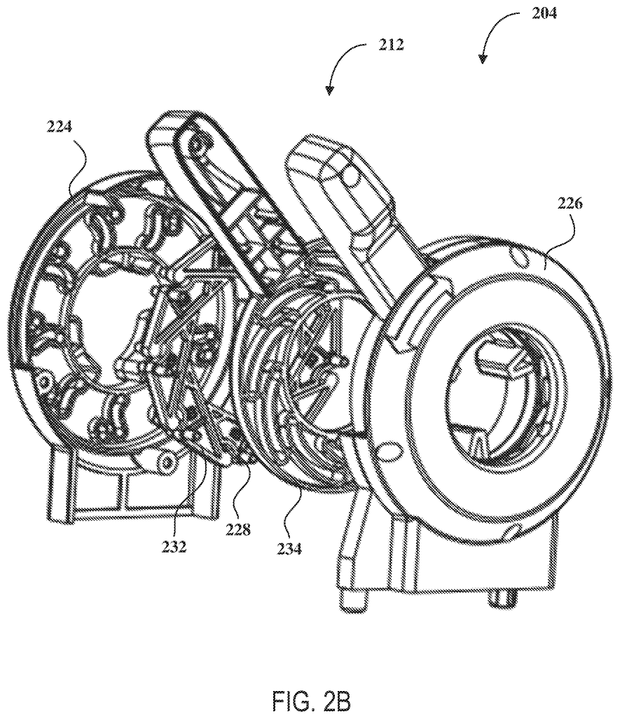

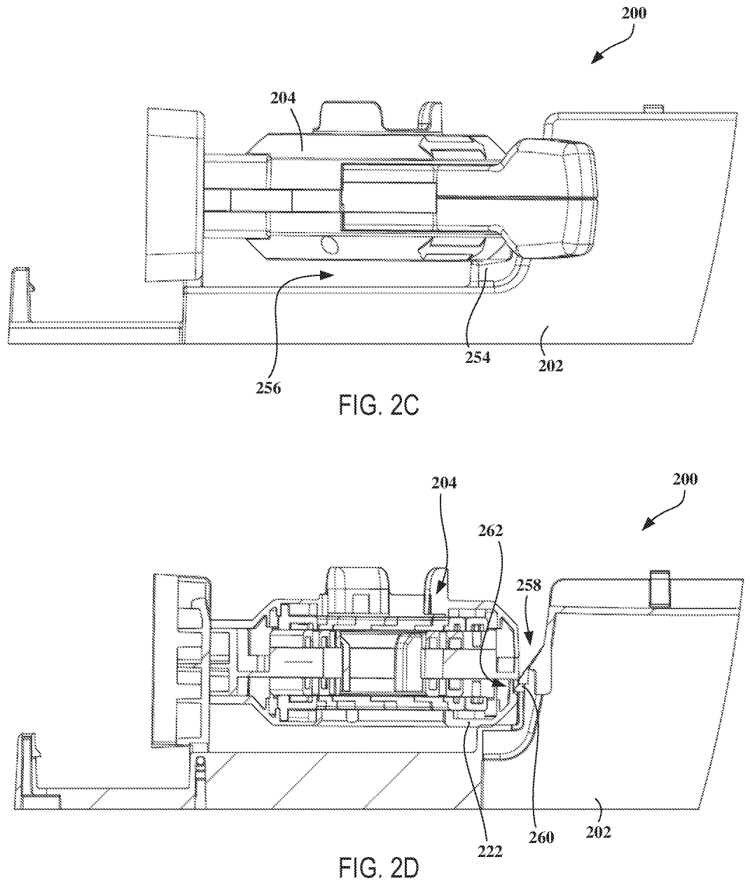

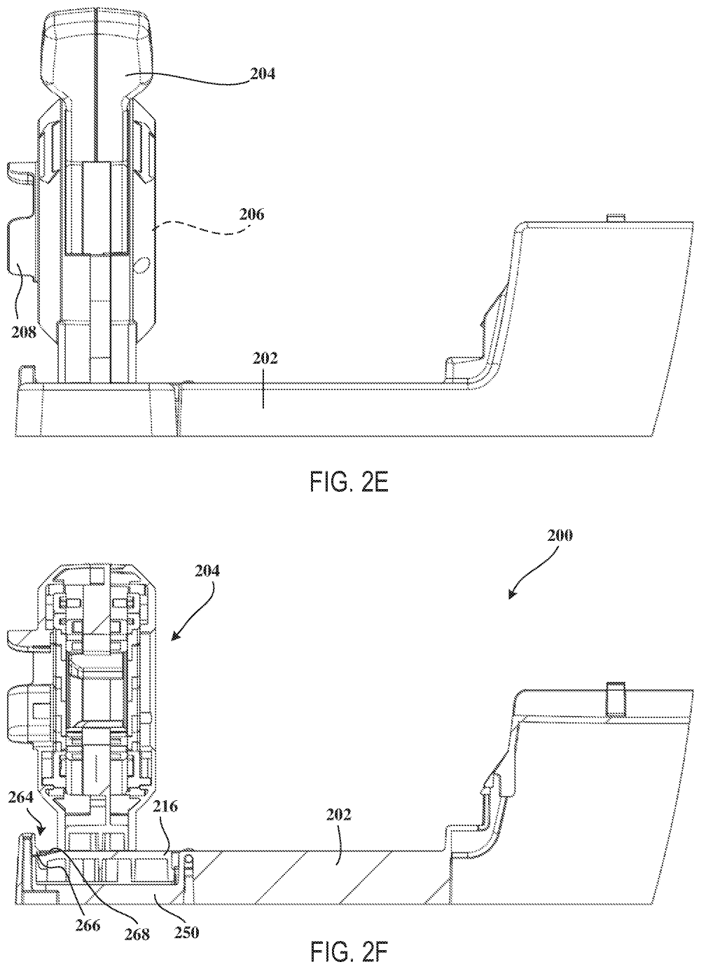

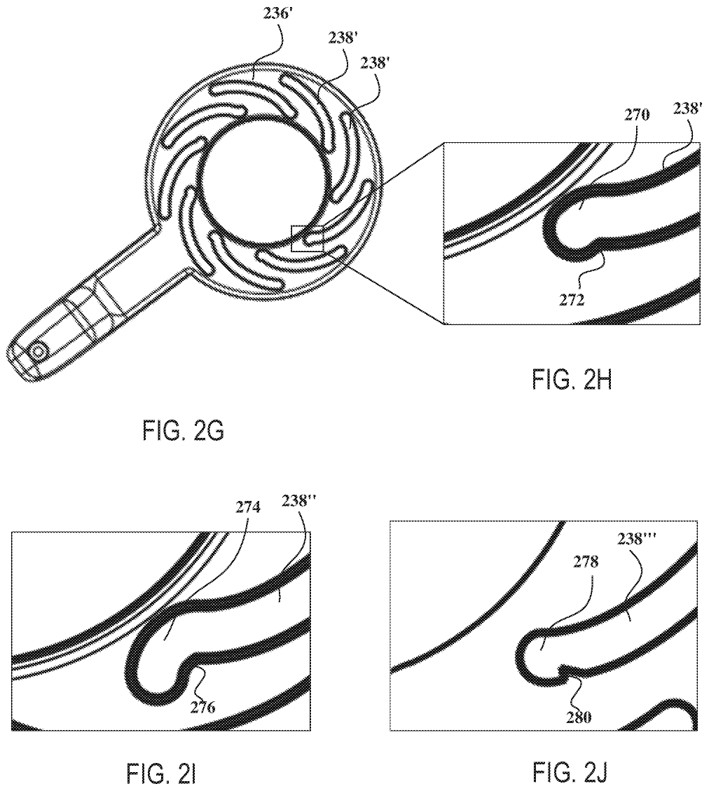

Referring now to FIGS. 2A to 2F, exemplary collapser system 200 for collapsing and loading a prosthetic device for delivery to a patient is described in further detail. FIG. 2A is an exploded view showing exemplary components of collapser system 200. As shown in FIG. 2A, collapser system 200 includes collapser 204 coupled to base 202. Internal components of collapser 204 are held in collapser housing 222, which may be formed from one or more pieces such as collapser covers 224, 226 as illustrated. Collapser cover 224 may include protrusions 208 to facilitate desirable alignment of the prosthetic device within collapser 204, as described above. Internal component housed between collapser covers 224, 226 may include first and second collapser sectors 228, 230, first and second annular bodies 234, 236, and/or central frame 240. As illustrated, central frame 240 may be in the middle of collapser 204 with first and second annular bodies 234, 236 on opposing sides of central frame 240. First and second collapser sectors 228, 230 are between first and second annular bodies 234, 236 and collapser covers 224, 226 respectively. Each of the components preferably defines a central orifice such that the components, when coupled together, define orifice 206.

First and second collapser sectors 228, 230 each may have a plurality of collapser sector segments 232. As illustrated, each collapser sector segment 232 may be angled plates that narrow at one end. The plurality of collapser sector segments 232 preferably rotate upon actuation such that they physically constrict the opening of orifice 206, thus reducing the size of orifice 206 from the first size to the second, smaller size. First and second collapser sectors 228, 230 each may have a plurality of components (e.g., posts) that extend outwardly to engage with the respective annular body and/or with respective collapser covers 224, 226. For example, each collapser sector segment 232 may include the protruding component at each of its ends.

First and second collapser annular bodies 234, 236 are each designed for relative rotation about an axis responsive to actuation. First and second collapser annular bodies 234, 236 each may have a plurality of curved and radially spaced apertures 238 to receive the components (e.g., posts) of first and second collapser sectors 228, 230, respectively. Collapser handle 214 further may be coupled to, or integrally formed with, first and second collapser annular bodies 234, 236. In this manner, movement of collapser handle 214 rotates annular bodies 234, 236, thereby causing collapser sectors 228, 230 to collapse on respective ends of the prosthetic device while the central portion of the prosthetic device is not contacted by collapser sectors 228, 230. In this manner, collapser system 200 permits collapsing select sections of the prosthetic device.

Central frame 240 may be coupled (e.g., fixedly coupled) to movable base 216. Central frame 240 may include arm supports 211 to contact anchoring arms of the prosthetic device during the collapsing process. Central frame 240 may be positioned between first and second collapser annular bodies 234, 236 in collapser housing 222. The components of collapser housing 222 may be secured via fasteners such as screws 242.

Moveable base 216 may be coupled to collapser 204, for example, via base pins 244, and moveably coupled to main base 202 via a hinge that may be formed of coupling hinge 246 and coupling pin 248. A portion of base 202, namely base extension 250, may extend under moveable base 216 while collapser 204 is in the second position, constituting a fixed surface upon which moveable base 216 may be temporarily located during collapsing and subsequent loading of a prosthetic device onto a delivery system. Moveable base 216 may include a recess on its lower surface to receive base extension 250 when in the second position. Base 202 further may include user instructions 252 that illustrate to the user (e.g., via text and/or pictures) step-by-step instructions for using collapser system 200.

FIG. 2B is another exploded view showing exemplary components of collapser system 200. As shown in FIG. 2B, collapser covers 224, 226 may include a plurality of curved and radially spaced internal tracks to receive the components (e.g., posts) of first and second collapser sectors 228, 230, respectively, to further facilitate secure collapsing. The plurality of curved and radially spaced internal tracks may correspond to the same shape and orientation as the corresponding curved and radially spaced apertures 238 of first and second collapser annular bodies 234, 236. Additionally, each of the plurality of collapser sector segments 232 of first and second collapser sectors 228, 230 may have a triangular shape (e.g., obtuse triangular shape) as shown in FIG. 2B.

FIG. 2C shows a front view of collapser system 200 in the initial position for top loading the prosthetic device. As shown in FIG. 2C, collapser system 200 further may include ledge 254 extending upwardly from base 202. Ledge 254 may be integrally formed with base 202. Ledge 254 preferably contacts and securely maintains collapser 204 in the first position. For example, ledge 254 may contact an upper portion of the housing of collapser 204 to hold collapser in the generally horizontal position for top loading of the prosthetic device. As shown, gap 256 may be defined between base 202 and collapser 204 in the first position, e.g., due to ledge 254.

FIG. 2D shows a cross-sectional view of collapser system 200 from FIG. 2C. As shown in FIG. 2D, collapser system 200 further may include lock 258 to maintain collapser 204 in the first position via coupling with base 202. Lock 258 may be positioned upwardly from ledge 254 as shown. Lock 258 may be releasable upon application of a predetermined force toward the second position. For example, a user can pull up on the handle of collapser 204 with a force at or greater than the predetermined force to release lock 258 for movement to the second position. Lock 258, however, is sufficiently secure such that collapser 204 should not unintentionally disengage from base 202, e.g., during transport, when the user retrieves and lifts collapser system 200 from its packaging/box, or during top loading. Alternatively or additionally, lock 258 may be released upon user actuation, e.g., pressing a button. Lock 258 may include flexible protrusion 260 extending from base 202 to engage collapser housing recess 262 at the top portion of collapser housing 222. Flexible protrusion 260 may deflect upon application of the predetermined force to release from collapser housing recess 262, thereby releasing lock 258.

FIG. 2E shows a front view of collapser system 200 in the position for loading the prosthetic device onto the delivery system. As shown by comparing FIG. 2E to FIG. 2C, collapser 204 has been moved from the first position to the second position. In the second position, orifice 206 is oriented in a different manner relative to base 202 than in the first position. For example, the longitudinal axis extending through orifice 206 is perpendicular to the elongated portion of base 202 in the first loading position while the longitudinal axis is parallel to the elongated position of base 202 in the second position. As such, the second position further may be substantially orthogonal to the first position to facilitate top loading of a prosthetic device into orifice 206 while collapser 204 is in the first position and then side loading of a delivery system through the same orifice 206 once collapser 204 has been moved to the second position.

FIG. 2F shows a cross-sectional view of collapser system 200 from FIG. 2E. As shown in FIG. 2F, collapser system 200 further may include base lock 264 to maintain collapser 204 in the second position via coupling with base 202. Base lock 264 may be positioned at an end of base 202 such as at an end of base extension 250 for releasable coupling with movable base 216. Base lock 264 is preferably released upon user actuation, e.g., pressing on the flexible protrusion to unsnap. Alternatively or additionally, base lock 264 may be releasable upon application of a predetermined force toward the first position (e.g., using the handle). Base lock 264 may include flexible protrusion 266 extending from base 202 (e.g., at base extension 250) to engage recess 268 at movable base 216. Flexible protrusion 266 may deflect upon user actuation to unsnap to release from recess 268, thereby releasing base lock 268. Preferably, base lock 264 is only unsnapped for prosthetic device recollapsing.

FIG. 2G illustrates annular body 236′ that may be used in collapser system 200 for self-locking the collapser when the orifice is in the collapsed position. Annular body 236′ corresponds to second annular body 236 (or first annular body 234) above although the plurality of radially spaced curved apertures 238′ of the first and second annular bodies self-lock when the first and second collapser sectors are in the position corresponding to the second, smaller size of the orifice. For example, the plurality of curved and radially spaced apertures may include features at an end (e.g., the proximal end) with a lock system that keep the collapser sectors closed at the minimum/collapsed diameter without the need to apply force on the collapser handle. This feature may be an abrupt change of curvature of the proximal end of the curved apertures.

FIGS. 2H to 2J illustrate various self-locking structures that may be incorporated into the collapser system. FIG. 2H illustrates self-lock 270 at the end of curved apertures 238′ where the curvature of the aperture changes at inflection point 272 to a tangential directional profile. As such, as the corresponding component (e.g., post) of first and second collapser sectors 228, 230 moves along curved aperture 238′, the component is redirected at self-lock 270 to lock collapser system 200 in place in the collapsed state without the need to press on the handle. FIG. 2I illustrates an alternative self-lock 274 at curved aperture 238″ where the curvature of the aperture changes at inflection point 276 to a reverse curvature profile as compared to the curvature before inflection point 276. FIG. 2J illustrates yet another alternative self-lock 278 at curved aperture 238′ where the end includes snap 280 that extends into curved aperture 238″ to stop the corresponding component (e.g., post) of first and second collapser sectors 228, 230 from moving back along curved aperture 238′″ without unsnapping snap 280 to disengage self-lock 278. As shown in FIG. 2J, the radius of curvature of curved aperture 238′ may be maintained along of the length of the aperture in this embodiment.

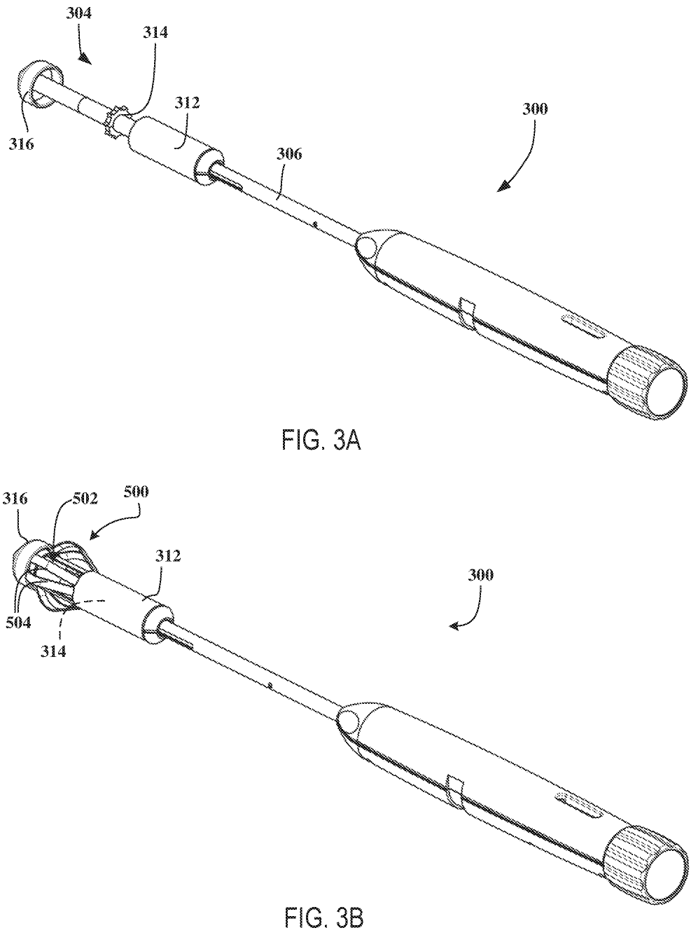

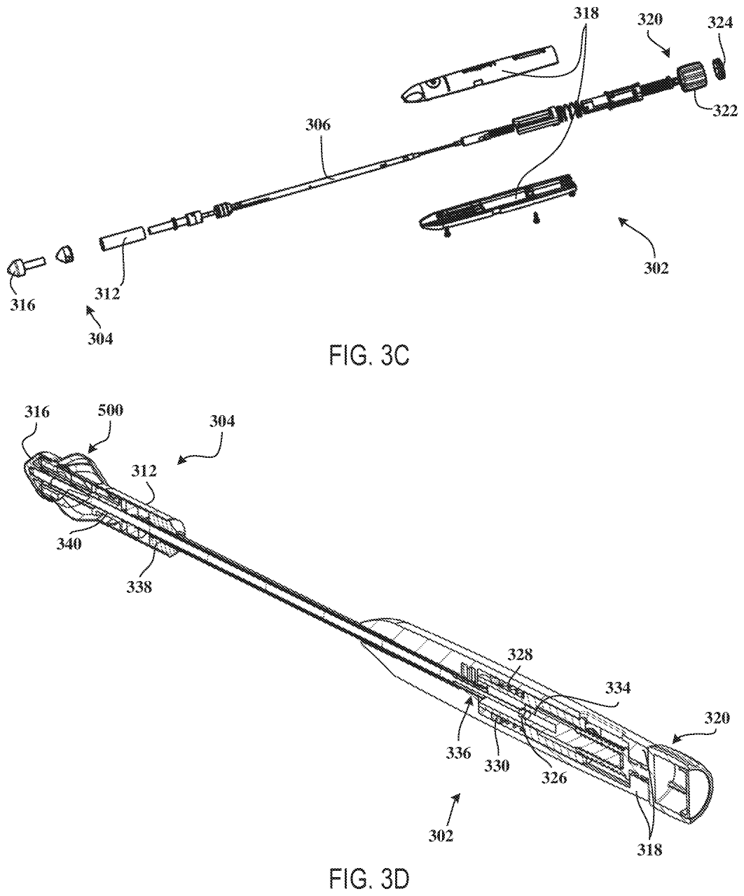

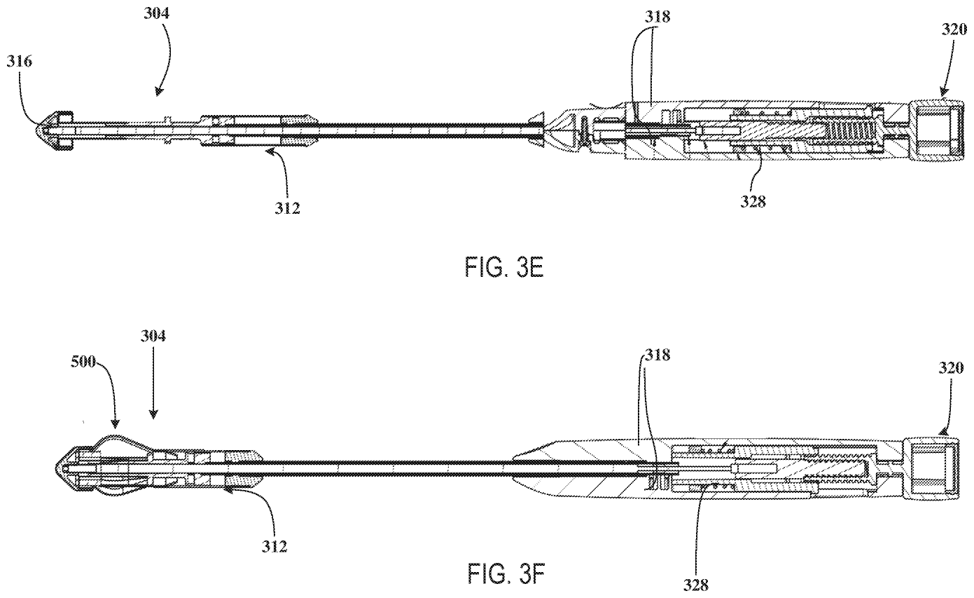

Referring now to FIGS. 3A to 3F, exemplary delivery system 300 for delivering exemplary prosthetic device 500 to a patient is provided. FIG. 3A shows a perspective view of delivery system 300 prior to collapsing and loading the prosthetic device thereon. As shown in FIG. 3A, distal portion 304 of delivery system 300 may include sheath 312, constraint formation 314, and distal tip 316 extending in a proximal-to-distal direction along shaft 306. Prior to collapsing and loading of a prosthetic device onto delivery system 300, distal portion 304 may receive a prosthetic device in an expanded state between sheath 312 and distal tip 316 such that sheath 312 does not contact the prosthetic device. Preferably, the frame of the prosthetic device is designed to be coupled to constraint formation 314.

FIG. 3B shows a perspective view of delivery system 300 having a collapsed and loaded prosthetic device 500 thereon. Illustratively, prosthetic device 500 is a prosthetic heart valve. As shown in FIG. 3B, prosthetic device 500 may include frame 502 with anchoring arms 504 that extend radially outward. Frame 502 may be structured to facilitate collapsing and expanding of prosthetic device 500, while anchoring arms 504 serve to position prosthetic device 500 in a preferable location at a delivery site of a patient (e.g., where a defective heart valve, such as the aortic heart valve, was located). Prosthetic device 500 may be collapsed and loaded onto delivery system 300 between constraint formation 314 and distal tip 316 such that sheath 312 longitudinally contracts with respect to distal tip 316, such that sheath 312 maintains one end (e.g., outflow end) of prosthetic device 500 in a collapsed state and distal tip 316 maintains the other end (e.g., inflow end) in the collapsed state, thereby permitting delivery to the patient for implantation.

FIG. 3C shows an exploded view of delivery system 300. As shown in FIG. 3C, proximal portion 302 may include delivery housing 318 and delivery actuator 320. Delivery actuator 320 is designed to be actuated by the user to cause corresponding movements of components at distal portion 304 of delivery system 300. Sheath 312 may, upon actuation, slide longitudinally along distal portion 304 from shaft 306 toward distal tip 316 to facilitate collapsing of a prosthetic device. Delivery actuator 320 further may include delivery knob 322, which may be buttressed against delivery cap 324. Delivery knob 322 may be turned by a user to facilitate actuation of delivery system 300, as described in further detail below.

FIG. 3D shows a cross-sectional, perspective view of delivery system 300 having a collapsed and loaded prosthetic device 500 thereon. As shown in FIG. 3D, proximal portion 302 of delivery system 300 further may include outer slider 326, spring 328, pusher 330, inner slider 332, inner tube 334, and outer tube 336, which together may be configured to, upon actuation via delivery actuator 320, cause the longitudinal distance between sheath 312 and distal tip 316 to be reduced until a point of engagement of corresponding ends of prosthetic device 500. Distal portion 304 further may include delivery bushing 338 and delivery support 340 to facilitate such actuation.

FIG. 3E shows a cross-sectional, front view of delivery system 300 prior to collapsing and loading the prosthetic device thereon.

FIG. 3F shows a cross-sectional, front view of delivery system 300 having a collapsed and loaded prosthetic device 500 thereon. As shown in FIGS. 3E and 3F, spring 328 may, upon actuation by delivery actuator 320, push longitudinally towards distal portion 304, thus effecting a corresponding movement by sheath 312 toward distal tip 316 until sheath 312 engages with prosthetic device 500. Further details on delivery system 300 may be found in the above-incorporated WO 2019/224581 (corresponding to U.S. Ser. No. 17/057,502).

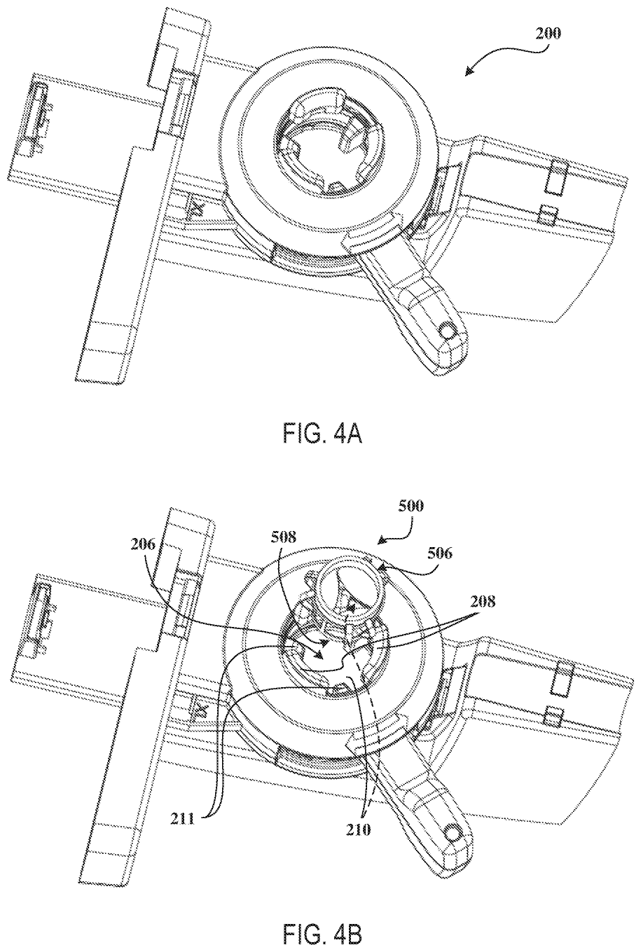

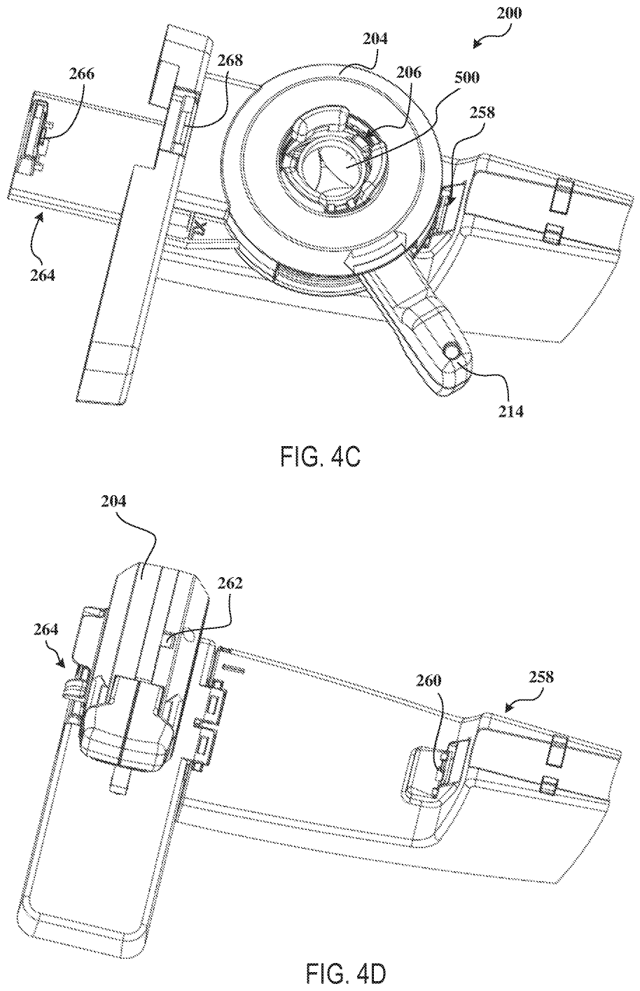

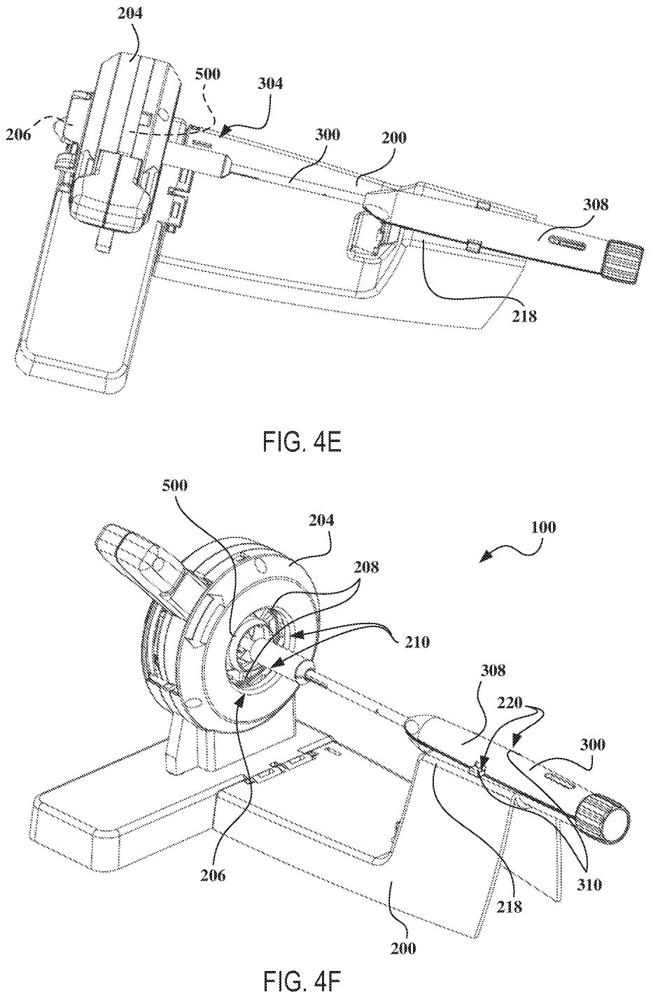

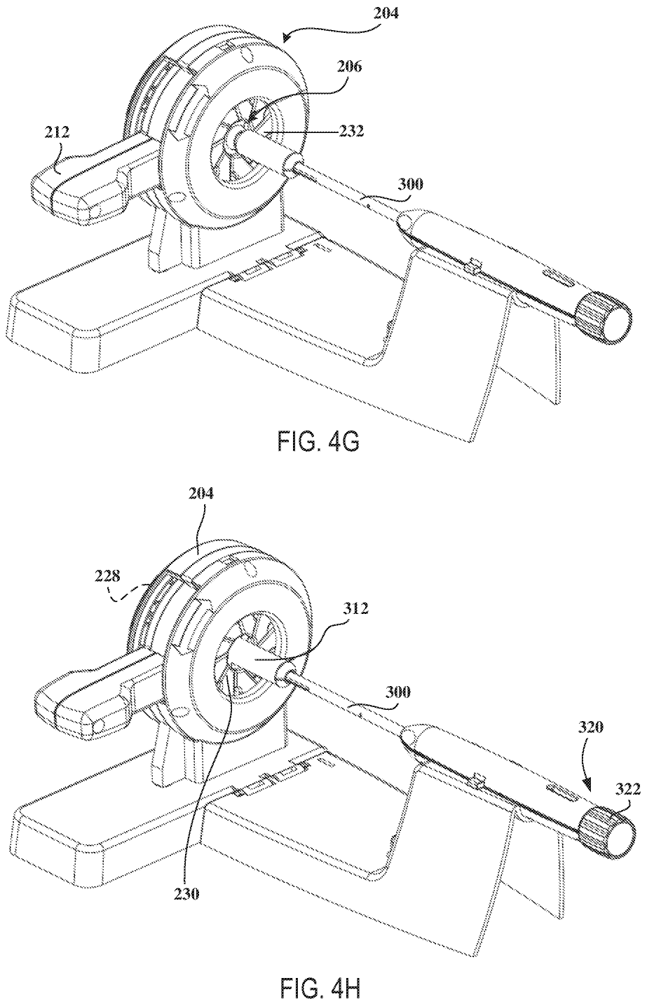

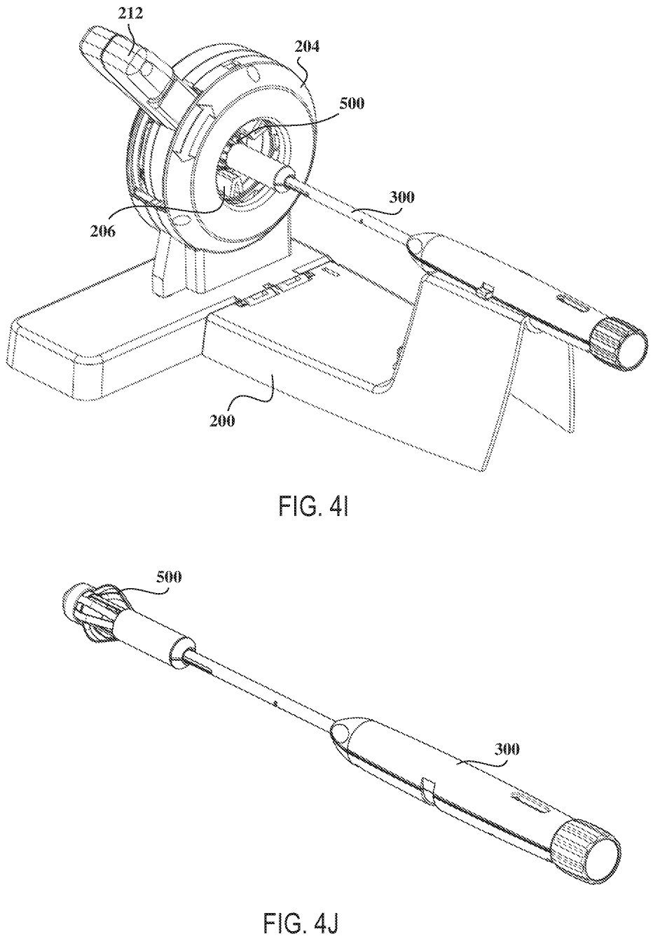

Referring now to FIGS. 4A to 4J, exemplary method for collapsing and loading a prosthetic device for delivery to a patient is provided. As shown in FIGS. 4A to 4J, system 100 may be provided with collapser system 200 and optionally delivery system 300. System 100 further may optionally include prosthetic device 500, which may be a prosthetic heart valve.

Referring to FIG. 4A, collapser system 200 is provided. Collapser system 200 may be in the first position, which may be an initial position. Collapser system 200 may be supplied in a package/kit of accessories provided with the prosthetic device to permit the clinician to implant the prosthetic device.

Prosthetic device 500 is provided as shown in FIG. 4B. For example, prosthetic device may be provided in a jar of sterile fluid that is opened in the operating room in a sterile environment. Advantageously, in some embodiments, prosthetic device 500 is able to be inserted in collapser system 200 without any additional components coupled thereto, such as a holder device. As shown in FIG. 4B, collapser system 200 may, in the first position, permit top loading of prosthetic device 500 in its expanded state into orifice 206. Protrusions 208 and voids 210 therebetween permit the user to align prosthetic device 500 for loading into orifice 206. For example, protrusions 208 extend into orifice 206 such that anchoring arms 504 that extend radially outward must be aligned with voids 210 to be inserted into orifice 206. Upon insertion, armature supports 211 contact respective anchoring arms 504 and may limit the depth of loading of prosthetic device 500 into orifice 206. Collapser system 200 may thus be configured to ensure that prosthetic device 500 is desirably aligned with respect to both axial orientation and positioning of inflow end 506 (which may include a plurality of prosthetic leaflets) and outflow end 508 of prosthetic device 500.

FIG. 4C is a perspective view of prosthetic device 500 loaded into orifice 206 of collapser system 200 prior to collapsing. As shown by comparing FIG. 4C to FIG. 4D, collapser 204 of collapser system 200 is moved from the first position to the second position shown in FIG. 4D. For example, the user can pull up on handle 214 of collapser system 200 at a force at or greater than a predetermined force to release lock 258 for movement to the second position. Upon application of such force, flexible protrusion 260 of lock 258 deflects to release from collapser housing recess 262, thereby releasing lock 258. In the second position, base lock 264 locks collapser 204 in place. For example, flexible protrusion 266 of base lock 264 extending from base 202 (e.g., at base extension 250) snaps into and engages recess 268 at movable base 216. Preferably, base lock 264 is only disengaged if necessary to later recollapse prosthetic device 500.

Referring to FIG. 4E delivery system 300 is provided. Delivery system 300 is positioned at collapser system 200 to permit collapser 204 to collapse prosthetic device 500 onto delivery system 300. For example, collapser 204 may permit side loading of delivery system 300 through orifice 206. Handle support 218 supports handle 308 of delivery system 300 while distal portion 304 is inserted through prosthetic device 500 positioned in orifice 206 for collapsing of prosthetic device 500.

FIG. 4F shows a perspective view of delivery system 300 positioned at collapser system 200 in a manner to permit collapsing of prosthetic device 500. As shown in FIG. 4F, collapser system 200 and corresponding delivery system 300 are positioned to facilitate precise collapsing and loading of prosthetic device 500 onto delivery system 300. Accordingly, collapser 204 may include a plurality of protrusions 208 that extend into orifice 206 and define a plurality of alignment voids 210 therebetween to facilitate initial alignment of prosthetic device 500 into orifice 206. Moreover, handle support 218 of collapser system 200 may include handle support aligners 220. Upon placement of handle 308 of delivery system 300 in handle support 218, handle support aligners 220 engage with handle aligners 310 of handle 308. As a result, delivery system 300 may be longitudinally and axially aligned in a desirable position for collapsing and loading of prosthetic device 500 situated in orifice 206.

FIG. 4G shows a perspective view of delivery system 300 positioned at collapser system 200 after actuation at actuator 212 to collapse prosthetic device 500. Upon user actuation, the size of orifice 206 is reduced from a first size (shown in FIG. 4F) to a second, smaller size (shown in FIG. 4G) to collapse prosthetic device 500 onto delivery system 300 while collapser 204 is in the second position. Collapser sector segments 232 are reoriented upon actuation to reduce the size of orifice 206 from the first size to the second, smaller size. For example, collapser sectors 228, 230 may collapse on respective ends of prosthetic device 500 while the central portion of prosthetic device 500 is not contacted by collapser sectors 228, 230.

While collapser 204 remains in the collapsed position shown in FIG. 4G, the user may actuate delivery system 300 to cause components at the distal portion of delivery system to move towards the delivery state. In some embodiments, actuating delivery system 300 moves proximal and distal components of delivery system 300 towards one another to hold first and second ends of the prosthetic device in the collapsed state.

For example, the user may actuate actuator 320 (e.g., by rotating knob 322) to cause sheath 312 to move distally and to cause the distal tip to move proximally, as shown in FIG. 4H. In this manner, sheath 312 may partially hold one end (e.g., outflow end) of prosthetic device 500 in the collapsed position and the distal tip may partially hold the other end (e.g., inflow end) in the collapsed position. However, in some embodiments, collapser 204, in the collapsed position shown in FIG. 4H, creates an opening smaller than sheath 312 and the distal tip, thereby preventing delivery system 300 to fully transitioning to the delivery state by restricting longitudinal motion of sheath 312 against collapser sectors 228, 230. Delivery system 300, however, is biased further (e.g., via a spring such as spring 328 of FIG. 3D) such that sheath 312 cannot run the full stroke as it stops against the collapser sector.

As explained above, collapser system 200 may include features to self-lock actuator 212 in the collapsed position shown in FIGS. 4G and 4H, such as features at ends of the plurality of curved and radially spaced apertures of the annular bodies. In this manner, the user need not apply force on the collapser handle while collapser 204 is in the collapsed position such that the user may free up his/her hands to perform other tasks such as actuating the delivery system to transition the delivery system towards the delivery state.

FIG. 4I shows a perspective view of delivery system 300 positioned at collapser system 200 after further actuation at actuator 212 to transition orifice 206 of collapser 204 from the collapsed position to the larger opening position. In some embodiments, the user manipulates actuator 212 to disengage the self-lock holding actuator 212 in the collapsed position such as be moving the component past the inflection point and/or unsnapping the component. As shown, prosthetic device 500 remains coupled to the distal portion of the delivery system in the collapsed state. In some embodiments, upon the further actuation, the collapser is transitioned from the smaller sized orifice to the larger sized orifice to cause the proximal and distal components of the delivery system to move closer towards one another to securely hold the prosthetic device in the collapsed state for delivery to the patient. Thus, the further actuation may fully transition the components at the distal portion of delivery system 300 to the delivery state. For example, pulling the collapser handle to open the sectors may allow the spring of delivery system 300 to fully unload, thereby allowing the sheath and distal tip to fully capture the ends of the prosthetic device.

FIG. 4J shows delivery system 300 having a collapsed and loaded prosthetic device 500 thereon after removal from collapser system 200. Prosthetic device 500 may then be delivered in the collapsed state using delivery system 300 to a target implantation site (e.g., the location of a defective heart valve, which may have been surgically removed). Once at the target implantation site, prosthetic device 500 may transitioned from the collapsed state to an expanded state to implant prosthetic device 500 at the target implantation site. The distal portion of the delivery system may then be withdrawn from the patient. Further expansion of prosthetic device 500 may be performed using balloon catheter 400. In this manner, for example, a prosthetic device may be implanted to treat a defective heart valve.

While various illustrative embodiments of the invention are described above, it will be apparent to one skilled in the art that various changes and modifications may be made therein without departing from the invention. The appended claims are intended to cover all such changes and modifications that fall within the true scope of the invention.

1. A system for collapsing a prosthetic device for delivery to a patient, the system comprising:
a base; and
a collapser coupled to the base, the collapser defining an orifice sized and shaped to receive a prosthetic device in an expanded state while the collapser is in a first position relative to the base, the collapser configured to move from the first position to a second position, wherein the orifice is oriented in a different manner relative to the base than the first position, the collapser further configured to reduce the size of the orifice from a first size to a second, smaller size to collapse the prosthetic device onto a delivery system while the collapser is in the second position, thereby preparing the prosthetic device for delivery to a patient in a collapsed state via the delivery system.

2. The system of claim 1, wherein the collapser further comprises an actuator configured to, upon actuation, cause the collapser to reduce the size of the orifice from the first size to the second, smaller size such that the prosthetic device is loaded in the collapsed state onto the delivery system.

3. The system of claim 2, wherein the actuator is configured to, upon further actuation, cause the collapser to increase the size of the orifice from the second, smaller size to the first size such that the prosthetic device remains collapsed onto the delivery system.

4. The system of claim 1, wherein the actuator comprises a handle.

5. The system of claim 1, further comprising a movable base coupled to the collapser and movably coupled to the base.

6. The system of claim 5, wherein a portion of the base extends under the movable base.

7. The system of claim 1, wherein the collapser, in the first position, permits top loading of the prosthetic device in the expanded state into the orifice and, in the second position, permits side loading of the delivery system through the orifice.

8. The system of claim 1, further comprising a lock configured to maintain the collapser in the first position via coupling with the base.

9. The system of claim 8, wherein the lock is releasable upon application of a predetermined force towards the second position.

10. The system of claim 8, wherein the lock comprises a flexible protrusion extending from the base to engage a recess at a top portion of a housing of the collapser, the flexible protrusion configured to deflect upon application of the predetermined force to release the recess.

11. The system of claim 1, further comprising a base lock configured to maintain the collapser in the second position via coupling with the base.

12. The system of claim 1, further comprising a handle support configured to support a handle of the delivery system while a distal portion of the delivery system is inserted in the orifice for collapsing of the prosthetic device.

13. The system of claim 12, wherein the handle support comprises alignment features configured to engage with corresponding features of the handle of the delivery system to longitudinally and axially align the delivery system during collapsing of the prosthetic device.

14. The system of claim 1, further comprising a ledge extending upwardly from the base, the ledge configured to contact and securely maintain the collapser in the first position.

15. The system of claim 1, wherein the first position is substantially orthogonal to the second position.

16. The system of claim 1, wherein the collapser comprises a housing configured to house first and second collapser sectors each having a plurality of collapser sector segments, the plurality of collapser sector segments configured to move upon actuation to reduce the size of the orifice from the first size to the second, smaller size.

17. The system of claim 16, wherein the collapser further comprises first and second annular bodies configured for relative rotation about an axis, the first and second annular bodies each having a plurality of radially spaced curved apertures to receive components of the first and second collapser sectors, respectively.

18. The system of claim 17, wherein the plurality of radially spaced curved apertures of the first and second annular bodies are configured to self-lock when the first and second collapser sectors are in a position corresponding to the second, smaller size.

19. The system of claim 17, wherein a handle is coupled to the first and second annular bodies.

20. The system of claim 17, wherein the collapser further comprises a collapser central frame configured to be positioned between the first and second annular bodies in the housing.

21. The system of claim 1, wherein the collapser further comprises a plurality of protrusions that extent into the orifice and define voids therebetween to facilitate alignment of the prosthetic device into the orifice.

22. The system of claim 1, wherein, in the second, smaller size, the collapser prevents components at a distal portion of the delivery system from fully transitioning to the delivery state.

23. The system of claim 1, further comprising the delivery system.

24. The system of claim 1, further comprising a balloon catheter configured to expand the prosthetic device after deployment from the delivery system.

25. The system of claim 1, further comprising the prosthetic device, wherein the prosthetic device comprises a prosthetic heart valve.

26. A method for collapsing a prosthetic device for delivery to a patient, the method comprising:
loading a prosthetic device into an orifice of a collapser while the collapser is in a first position relative to a base;
moving the collapser from the first position to a second position while the base remains in place;
inserting a distal portion of a delivery system through the prosthetic device loaded into the orifice; and
collapsing the prosthetic device onto the distal portion of the delivery system via the collapser to prepare the prosthetic device for delivery to a patient in a collapsed state via the delivery system.

27. The method of claim 26, further comprising:
after collapsing the prosthetic device, actuating the delivery system to move proximal and distal components of the delivery system towards one another to hold proximal and distal ends of the prosthetic device in the collapsed state; and
transitioning the collapser from the smaller sized orifice to a larger sized orifice such that the prosthetic device remains coupled to the distal portion of the delivery system in the collapsed state.

28. The method of claim 27, wherein transitioning the collapser from the smaller sized orifice to the larger sized orifice causes the proximal and distal components of the delivery system to move closer towards one another to securely hold the prosthetic device in the collapsed state for delivery to the patient.
